(19) United States
(12) Patent Application Publication
(10) Pub. No.: US 2019/0201611 A1
(43) Pub. Date: Jul. 4, 2019

(54) Blood Purification System Using UV Radiation

(71) Applicant: AIRx Medical, Inc., Pleasanton, CA (US)

(72) Inventors: Clarisa Tate, Mountain House, CA (US); Jeffrey Cohen, Pleasanton, CA (US)

(73) Assignee: AIRx Medical, Inc., Pleasanton, CA (US)

(21) Appl. No.: 15/860,923

(22) Filed: Jan. 3, 2018

(51) Int. Cl. A61M 1/36; A61L 2/00

(52) U.S. Cl. CPC A61M 1/3681; A61M 2205/053; A61L 2/0047

(57) ABSTRACT

An apparatus for reducing pathogens in blood. An infusing blood source provides a supply of blood at a selectable flowrate. A blood transportation line transports the blood from the infusion blood source. A UV-passing cuvette receives the blood from the blood transportation line. An irradiation chamber has a clamp for fixing the UV-passing cuvette at a predetermined gap from the UV radiation source for exposing the blood within the UV-passing cuvette directly to UV radiation. A radiation recycling coating is formed on surfaces of the irradiation chamber for reflecting and recycling the UV radiation to expose the blood indirectly to the UV radiation. The predetermined gap and the flow rate are selected so that the direct and indirect exposure of the blood to the UV radiation kills pathogens in the blood while limiting the loss of healthy blood cells and blood constituents due to the UV radiation and heat generated by the UV radiation source.

### TECHNICAL FIELD

This invention relates generally to a blood purification system and, more specifically, relates to a blood purification system that uses UV radiation to irradiate blood to kill pathogens.

### BACKGROUND

This section is intended to provide a background or context to the invention disclosed below. The description herein may include concepts that could be pursued, but are not necessarily ones that have been previously conceived, implemented or described. Therefore, unless otherwise explicitly indicated herein, what is described in this section is not prior art to the description in this application and is not admitted to be prior art by inclusion in this section.

There have been prior attempts at blood purification using UV exposure, with an early U.S. Pat. No. 2,308,516; issued to Knott “Method and Means for Irradiating Blood”; where a quantity of blood is removed from the patient, passed through a chamber where the blood is exposed to ultra-violet radiation and returned to the patient. Other examples of prior attempts at using UV radiation for blood purification include DE 4330189 A1, U.S. Pat. Nos. 6,113,566 A, 9,265,876 B1, US 20030086817 A1, U.S. Pat. Nos. 7,547,391 B2, 8,778,263 B2, CN 2162248 Y, U.S. Pat. Nos. 6,193,681 B1, 6,190,608 B1, EP 1902740 A1, U.S. Pat. No. 7,229,427 B2, US 20040186412 A1, and U.S. Pat. No. 8,883,409 B1.

However, the generation of UV radiation results in a significant generation of heat. Heating of the blood being purified has detrimental effects, causing the reduction in the number of healthy red and white blood cells, and the diminishment or damage to other healthy blood constituents.

There is a need for a blood purification system that balances the exposure of the healthy blood cells and other healthy blood constituents to radiation and heat while providing for the effective killing off of pathogens in the blood through the exposure to the radiation.

### SUMMARY OF THE INVENTION

This section is intended to include examples and is not intended to be limiting.

In accordance with an aspect of the present invention, an apparatus is provided for reducing pathogens in blood. An infusing blood source provides a supply of blood at a selectable flowrate. A blood transportation line transports the blood from the infusion blood source. A UV-passing cuvette receives the blood from the blood transportation line. The UV-passing cuvette is held in an irradiation chamber having a UV radiation source. The irradiation chamber has a clamp for fixing the UV-passing cuvette at a predetermined gap from the UV radiation source for exposing the blood within the UV-passing cuvette directly to UV radiation emitted from the radiation source. A radiation recycling coating is formed on surfaces of the irradiation chamber for reflecting and recycling the UV radiation to expose the blood indirectly to the UV radiation emitted from the radiation source. In accordance with the inventive blood purification apparatus, the predetermined gap and the flow rate are selected so that the direct and indirect exposure of the blood to the UV radiation kills pathogens in the blood while limiting the loss of healthy blood cells and blood constituents due to the UV radiation and heat generated by the UV radiation source.

In accordance with another aspect of the present invention, a method is provided for reducing pathogens in blood. Blood is supplied at a selectable flowrate from an infusion blood source through a blood transportation line to a UV-passing cuvette. An irradiation chamber is provided having a UV radiation source. The UV-passing cuvette is fixed at a predetermined gap from the UV radiation source. The blood within the UV-passing cuvette is exposed directly to UV radiation emitted from the radiation source. The blood within the UV-passing cuvette is also exposed indirectly to UV radiation emitted from the radiation source. The irradiation chamber has a radiation recycling coating formed on surfaces of the irradiation chamber for reflecting and recycling the UV radiation to expose the blood indirectly to the UV radiation emitted from the radiation source. The predetermined gap and the flow rate are selected so that the direct and indirect exposure of the blood to the UV radiation kills pathogens in the blood while limiting the loss of healthy blood cells and blood constituents due to the UV radiation and heat generated by the UV radiation source.

In accordance with another aspect of the present invention, an apparatus is provided for reducing pathogens in blood. An infusing blood source provides a supply of blood at a selectable flowrate. A blood transportation line transports the blood from the infusion blood source. A UV-passing cuvette receives the blood from the blood transportation line. The UV-passing cuvette is held in a first irradiation chamber having a first UV radiation source. The first irradiation chamber has a clamp for fixing the UV-passing cuvette at a predetermined gap from the first UV radiation source for exposing the blood within the UV-passing cuvette directly to UV radiation emitted from the first radiation source. A radiation recycling coating is formed on surfaces of the first irradiation chamber for reflecting and recycling the UV radiation to expose the blood indirectly to the UV radiation emitted from the radiation source. At least a second irradiation chamber is provided having a second UV radiation source, second clamp for fixing a second UV-passing cuvette at the predetermined gap from the second UV radiation source and the radiation recycling coating. The predetermined gap and the flow rate are selected so that the direct and indirect exposure of the blood to the UV radiation kills pathogens in the blood while limiting the loss of healthy blood cells and blood constituents due to the UV radiation and heat generated by the UV radiation source.

### BRIEF DESCRIPTION OF THE DRAWINGS

In the attached Drawing Figures:

FIG. 1 is a perspective view of a lower intensity embodiment of the inventive blood purification system;

FIG. 2 shows light chambers of the embodiment of the inventive blood purification system shown in FIG. 1;

FIG. 3 shows a UV radiation generating bi-pin straight bulb used in the embodiment of the inventive blood purification system shown in FIG. 1;

FIG. 4 illustrates a gap between a UV-passing cuvette and the bulb used in the embodiment of the inventive blood purification system shown in FIG. 1;

FIG. 5 illustrates a routine volume disposable cuvette used with the inventive blood purification system;

FIG. 6 illustrates a small volume disposable cuvette used with the inventive blood purification system;

FIG. 7 illustrates a male to male luer PVC subassembly;

FIG. 8 illustrates a 6″ male to female luer PVC extension subassembly;

FIG. 9 illustrates a 3″ male to female luer PVC extension subassembly;

FIG. 10 illustrates a routine volume disposable assembly for use with the inventive blood purification system;

FIG. 11 illustrates a 3-way stopcock for use with the disposables assembly;

FIG. 12 illustrates another 3-way stopcock for use with the disposables assembly;

FIG. 13 illustrates a routine volume disposables assembly for veterinary use of the inventive blood purification system;

FIG. 14 illustrates a small volume disposables assembly for veterinary use of the inventive blood purification system;

FIG. 15 is a perspective view of the embodiment of the inventive blood purification system shown in FIG. 1 including the disposables assembly ready for connection to pump and IV line;

FIG. 16 shows the full set up of the disposables assembly;

FIG. 17 shows an embodiment of the inventive blood purification system for use in exposing small volumes of blood with UVC radiation;

FIG. 18 shows an infusion syringe of an infusion configuration of the inventive blood purification system;

FIG. 19 shows a receiving syringe of a receiving configuration of the inventive blood purification system;

FIG. 20 illustrates a portable configuration of the inventive blood purification system;

FIG. 21 illustrates the portable configuration of the inventive blood purification system housed in a portable housing;

FIG. 22 is a perspective view of a higher intensity embodiment of the inventive blood purification system;

FIG. 23 shows light chambers of the embodiment of the inventive blood purification system shown in FIG. 22;

FIG. 24 shows a UV radiation generating four pin twin bulb used in the embodiment of the inventive blood purification system shown in FIG. 22; and

FIG. 25 illustrates a gap between a UV-passing cuvette and the bulb used in the embodiment of the inventive blood purification system shown in FIG. 22.

### DETAILED DESCRIPTION OF THE DRAWINGS

All of the embodiments described in this Detailed Description are exemplary embodiments provided to enable persons skilled in the art to make or use the invention and not to limit the scope of the invention which is defined by the claims.

The exemplary embodiments herein describe apparatus and techniques for using UV irradiation for purifying blood. The blood is exposed to UV radiation to kill pathogens while limiting the destruction of healthy blood cells and other healthy blood constituents. Additional description of these techniques is presented after a system into which the exemplary embodiments may be used is described.

The inventive system 10 uses a high-grade quartz cuvette 12 tube that allows blood to flow through an irradiation chamber 14 that includes a specular grade aluminum coating to increase the exposure of the blood in the tube to irradiation, e.g., UV-C radiation. A smaller gap 18 between the quartz cuvette 12 tube containing the blood and the UV-C radiation source 20 means that (according to the inverse square law) the blood is exposed to a higher direct dose of UV-C than it otherwise would receive if the gap 18 is larger. The inventive system 10 uses flexible tubing 22 that is not necessarily UV transmissive to bring the blood to and from the irradiation chamber 14. The inventive system 10 may be configured for use so that the same patient that provides the blood also receives back the purified blood. The inventive system 10 can be used with small animal studies because it is effective with small quantities of blood. The inventive system 10 is optimized to kill harmful bacteria and viruses while minimizing damage to healthy blood cells. Possible use of chillers before and/or after blood flows through irradiation chamber 14 keep blood from heating up and retaining harmful excess heat which may kill off healthy blood cells.

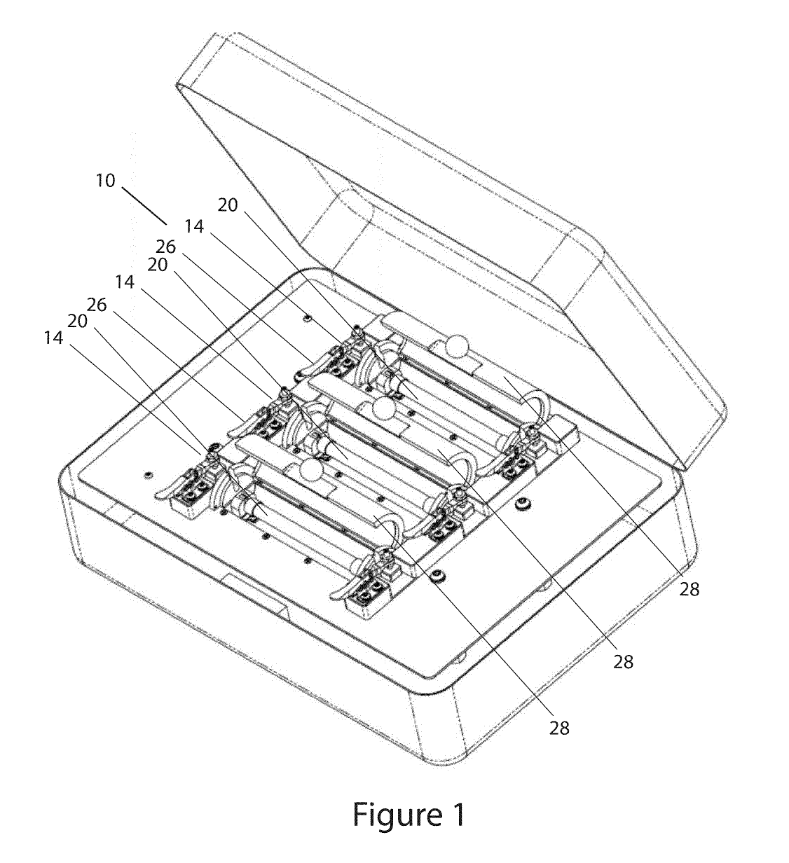

FIG. 1 is a perspective view of a lower intensity embodiment of the inventive blood purification system. In accordance with an exemplary embodiment, an apparatus is provided for reducing pathogens in blood. An infusing blood source provides a supply of blood at a selectable flowrate. A blood transportation line 24 transports the blood from the infusion blood source. A UV-passing cuvette 12 receives the blood from the blood transportation line 24. The UV-passing cuvette 12 is held in an irradiation chamber 14 having a UV radiation source 20.

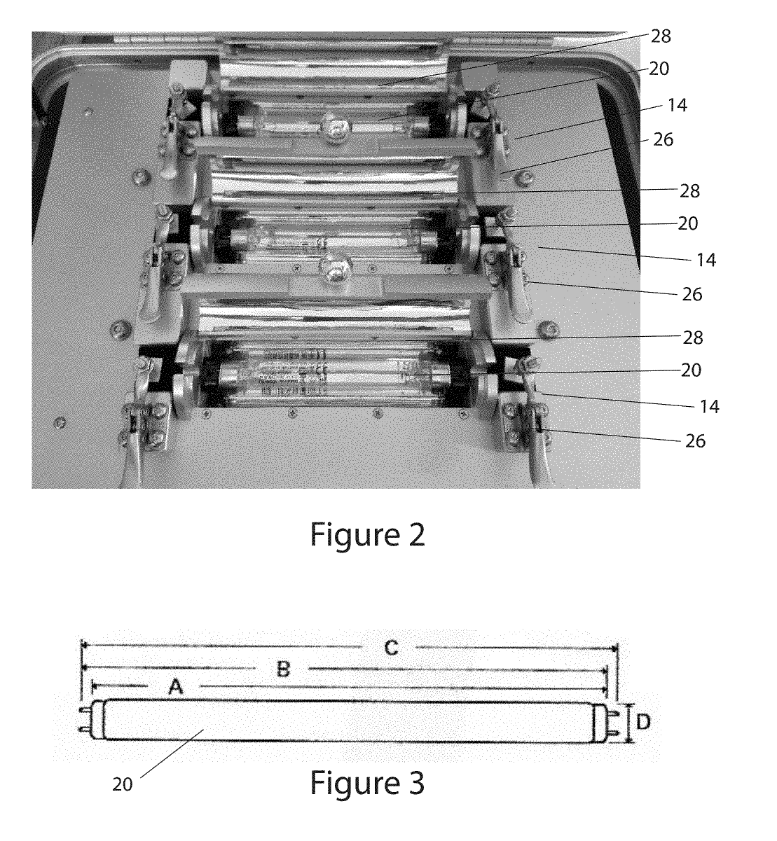

FIG. 2 shows light chambers 14 14 of the embodiment of the inventive blood purification system shown in FIG. 1 with clamps 26 with cushion, chamber cover 28, an internal chamber 14 and the installed bulbs 20. The irradiation chamber 14 has a clamp 26 for fixing the UV-passing cuvette 12 at a predetermined gap 18 from the UV radiation source 20 for exposing the blood within the UV-passing cuvette 12 directly to UV radiation emitted from the radiation source 20. A radiation recycling coating is formed on surfaces of the irradiation chamber 14 for reflecting and recycling the UV radiation to expose the blood indirectly to the UV radiation emitted from the radiation source 20. In accordance with the inventive blood purification apparatus, the predetermined gap 18 and the flow rate are selected so that the direct and indirect exposure of the blood to the UV radiation kills pathogens in the blood while limiting the loss of healthy blood cells and blood constituents due to the UV radiation and heat generated by the UV radiation source 20.

FIG. 3 shows UV radiation generating bi-pin straight bulb used in the embodiment of the inventive blood purification system shown in FIG. 1. In accordance with this exemplary embodiment, straight bulbs are used as the UV radiation source. The bulbs may be, for example, 4 watt or 9 watt 6 inch UV generating bulbs. The intensity or wattage of the bulbs is selected so that by controlling the predetermined gap 18 and the flow rate the direct and indirect exposure of the blood to the UV radiation is effective to kill the pathogens while limiting the loss of healthy blood cells and blood constituents due to the UV radiation and heat generated by the UV radiation source. The predetermined gap 18 may be, for example, in the range of 4 mm or 0.157 inches.

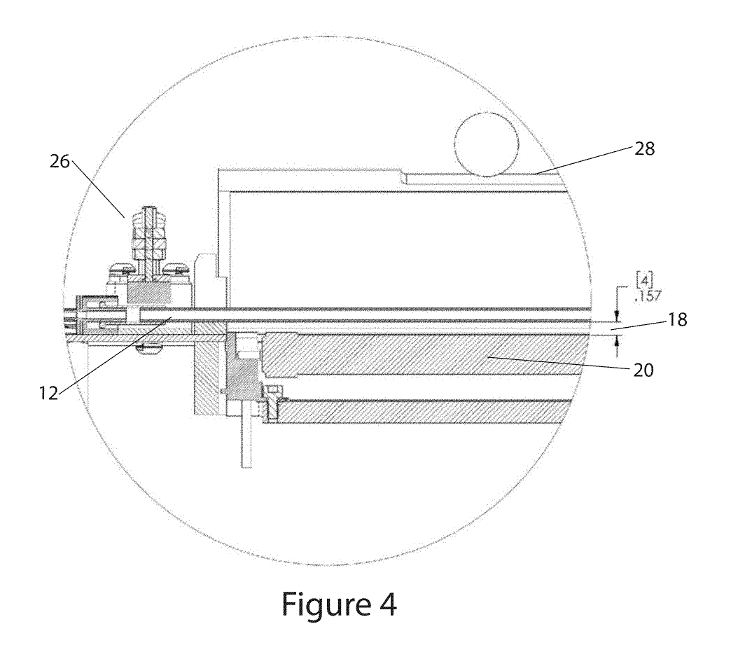

FIG. 4 illustrates a gap 18 between a UV-passing cuvette 12 and the bulb (radiation source) used in the embodiment of the inventive blood purification system shown in FIG. 1. The smaller the gap 18, the more intense the irradiation received directly from the bulb. Spherical aluminum, or other UV reflecting enhanced coating, may be provided as a high efficiency reflective surface. The UV reflecting enhanced coating is provided to recycle the light emitted from the bulb that is not irradiated directly from the bulb to the blood in the chamber. The recycling of the UV light provided by the bulb, along with the minimizing of the gap 18 between the bulb and the blood, increases the efficiency of the system. This increased efficiency provides a beneficial cycle that enables faster throughput, higher UV radiation dosing to kill pathogens, and lower thermal exposure to the blood.

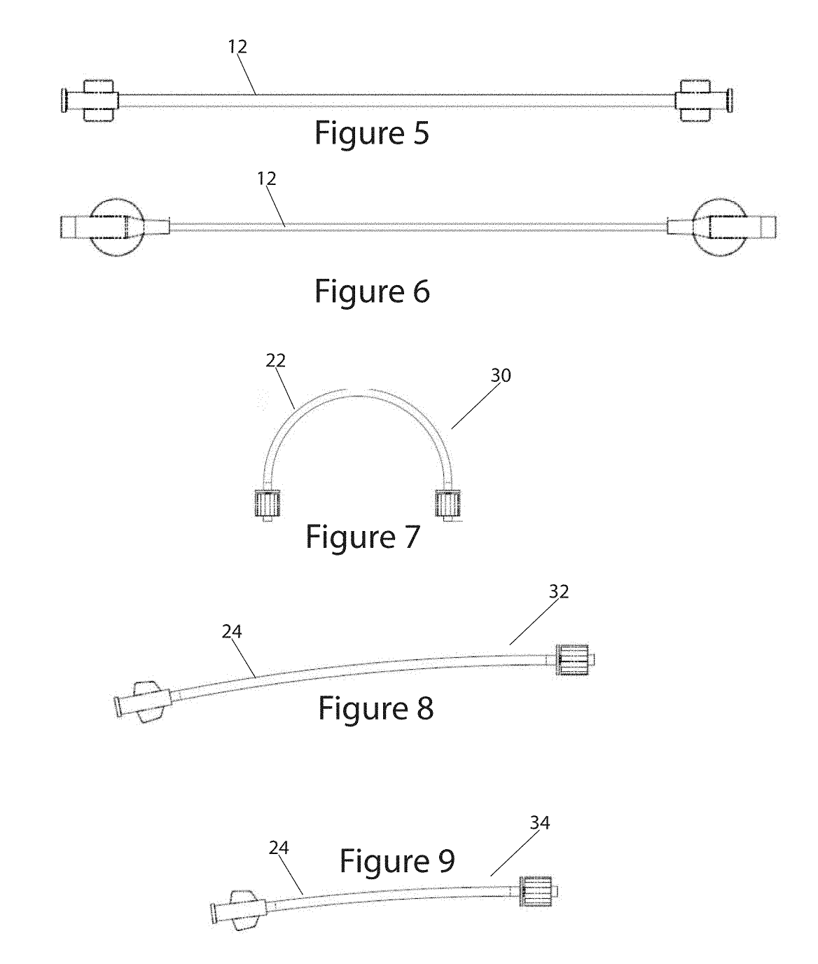

FIG. 5 illustrates a routine volume disposable cuvette 12 used with the inventive blood purification system, where the routine volume is sufficient for human use. FIG. 6 illustrates a small volume disposable cuvette 12 used with the inventive blood purification system, where the small volume is sufficient for small animals. The option of choosing between the routine volume disposable cuvette 12 and the small volume disposable cuvette 12 further enhances the efficiency and utility of the inventive blood purification system.

FIG. 7 illustrates a male to male luer PVC subassembly 30. FIG. 8 illustrates a 6″ male to female luer PVC extension subassembly 32. FIG. 9 illustrates a 3″ male to female luer PVC extension subassembly 34. The UV-passing cuvette 12 may be a disposable quartz tube, and the blood transportation line 24 may comprise a disposable flexible tubing 22 for connecting with the UV-passing cuvette 12.

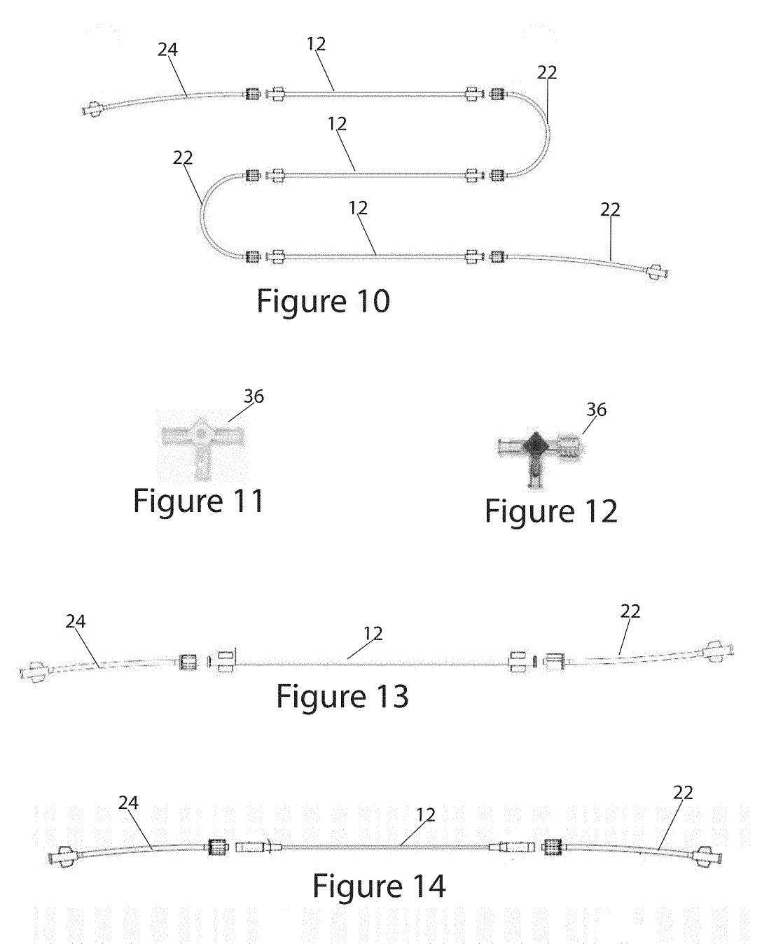

FIG. 10 illustrates a routine volume disposable blood irradiation assembly for use with the inventive blood purification system. A routine volume disposable assembly set for use with an embodiment of the inventive blood purification system consists of (3) 3×4 mm cuvette 12 subassembly (206 mm length), (2) male to male luer PVC subassembly, and (2) 6″ male to female luer PVC extension subassemblies. FIG. 11 illustrates a 3-way stopcock 36 for use with the disposables assembly. FIG. 12 illustrates another 3-way stopcock 6 for use with the disposables assembly.

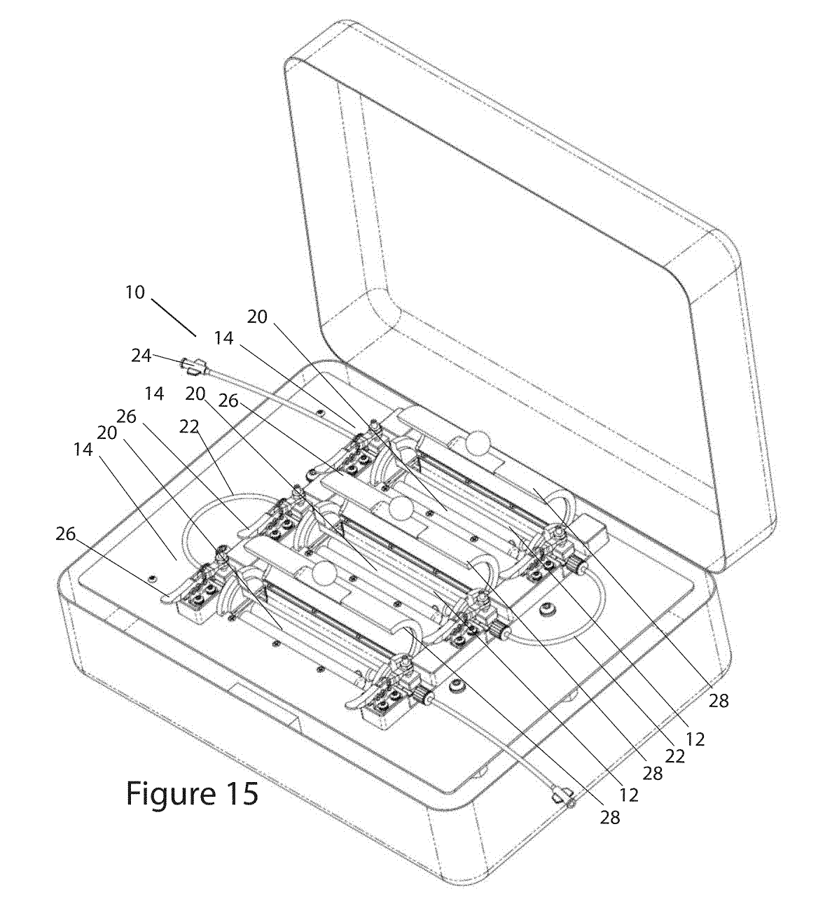

FIG. 13 illustrates a small volume disposable blood irradiation assembly for veterinary use of the inventive blood purification system using a 1×2 mm cuvette 12 subassembly. FIG. 14 illustrates a small volume disposable blood irradiation assembly for veterinary use of the inventive blood purification system using a 1×2 mm cuvette 12 subassembly. A small volume disposable assembly set for use with an embodiment of the inventive blood purification system consists (1) 3×4 mm 206 mm length cuvette 12 subassembly, or, (1) 1×2 mm 206 mm length cuvette 12 subassembly and (2) 3″ male to female luer PVC extension subassemblies.

FIG. 15 is a perspective view of the embodiment of the inventive blood purification system shown in FIG. 1 including the disposable blood irradiation assembly as the blood transportation line 24 ready for connection to pump and IV line. A light box and disposables are combined into a configured blood purification system, ready for connection to a pump and IV line.

The irradiation chamber 14 may further include a cover 28 having the radiation recycling coating for further recycling the radiation emitted from the radiation source 20 to expose the blood indirectly to the UV radiation emitted from the radiation source.

At least one additional similarly configured irradiation chamber 14 may be provided so that the blood is purified by flowing through two or more irradiation chambers 14. The use of two or more irradiation chambers 14 provides for the killing of the pathogens while limiting the destruction of the healthy blood cells and other healthy blood constituents. The predetermined gap 18 between the blood in the UV-passing cuvette 12 and the flow rate can be controlled so that the direct and indirect exposure of the blood to the UV radiation as the blood flows through the two or more irradiation chambers 14 kills the pathogens while limiting the loss of healthy blood cells and blood constituents due to the UV radiation and heat generated by the UV radiation source. The purified blood may be reinfused into the patient.

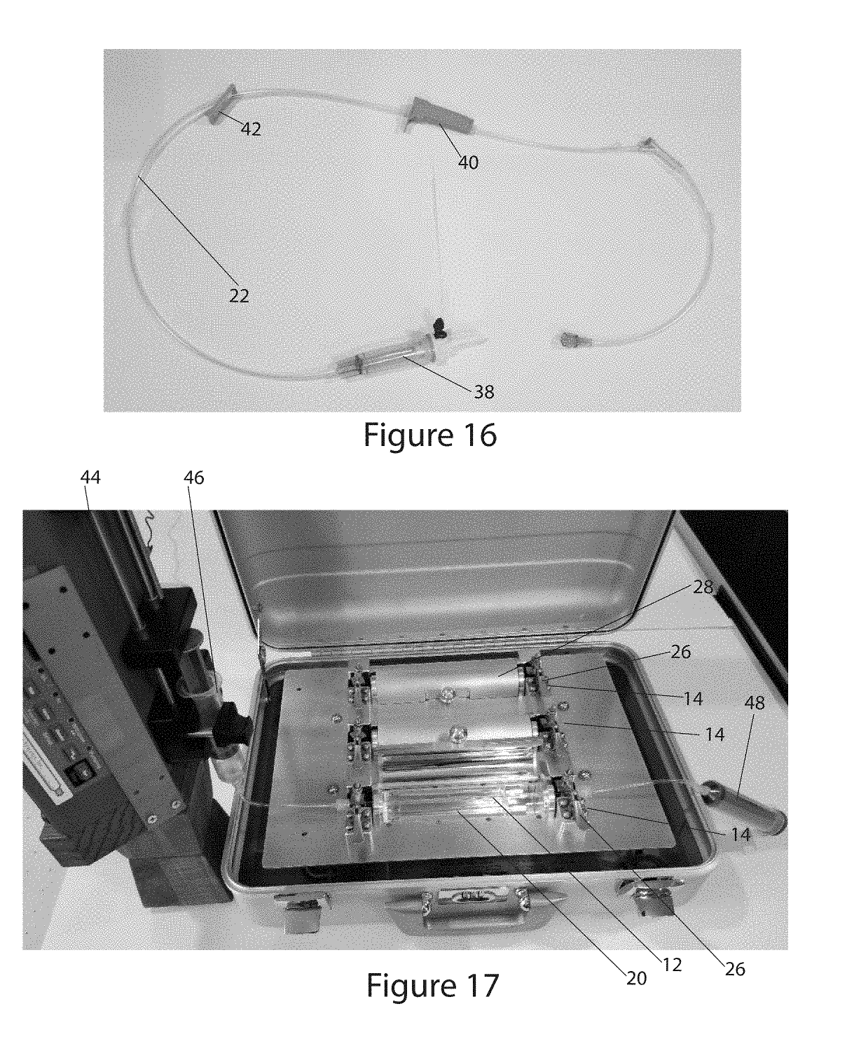

FIG. 16 shows the full set up of an embodiment of disposable blood irradiation assembly for use with the inventive blood purification system. In accordance with this embodiment, the constituent parts include a meter-long PVC tubing 22 on which is located a syringe injection port, and a male luer lock at the end that connects to the disposable blood irradiation assembly. A spike 38 with chamber may be provided for use with a blood bag that can be spiked. A roller clamp 40 and clamp 42 may be provided to control the flow of blood from the supply (e.g., a spiked blood bag). The disposable flexible tubing 22 may have a length of substantially one meter to facilitate moving the blood back and forth through the irradiation chamber.

The blood transportation line 24 may include a blood bag spike 38 for communicating with a blood bag. The infusing blood source may include a blood bag for containing the blood and a roller pump disposed on the blood transportation line 24 for pumping the blood from the blood bag through the blood transportation line 24 and to the UV-passing cuvette 12.

FIG. 17 shows an embodiment of the inventive blood purification system for use in exposing small volumes of blood with UVC radiation. A highly reflective surface, such as specular aluminum, may be provided to recycle the radiation emitted by a radiation source 20 bulb disposed within a radiation chamber. The inventive blood purification system may include a number of radiation chambers 14. Each radiation chamber includes clamps 26 for holding the quartz cuvette 12 subassembly in position so that blood flowing through the quartz cuvette 12 subassembly is irradiated by the UVC radiation emitted from the radiation source 20 bulb. Each radiation chamber may include a radiation recycling enhancing cover. The radiation recycling enhancing cover 28 can have a semi-circular cross section and closes over the radiation source 20 bulb so that blood flowing within quartz cuvette 12 subassembly receives radiation directly emitted from the radiation source 20 bulb and also receives radiation indirectly reflected from the radiation recycling enhancing cover 28.

The inventive blood purification system is configured and dimensioned to allow for use in exposing different volumes of blood depending on the intended use requirement. For example, small volumes of blood (e.g., about 4 mL) can be radiated with UVC radiation to kill off pathogens such as bacteria and viruses, while maintaining the health of blood cells and the biological performance of other healthy blood constituents.

In accordance with an embodiment of the inventive blood purification system, a syringe pump 44 is used to cause non-purified blood to flow through the disposable blood irradiation assembly. The syringe pump 44 may deliver, for example, 65mL/hr of the non-purified blood to the disposable blood irradiation assembly. The infusing syringe 46 used to hold the non-purified blood may be, for example, a 35 mL monoinject syringe with male luer lock can be used with PVC extensions that are cut at approximately 10 cm length so that during use the PVC extensions form a loop to prevent kinking. The PVC extensions are cut prior to assembly with male and female luers at either end.

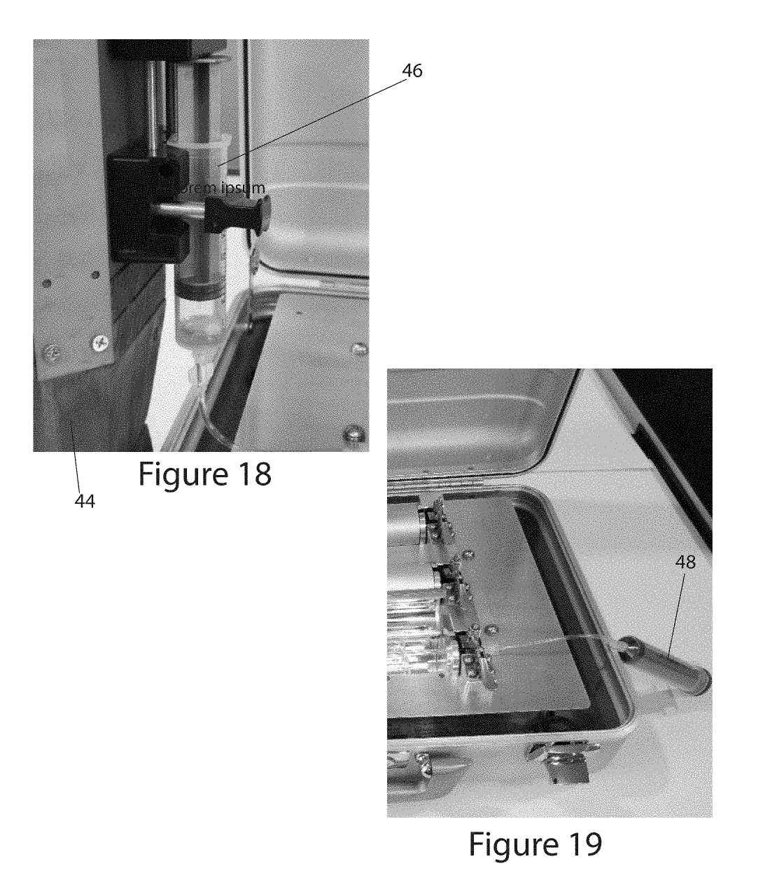

FIG. 18 shows an infusing syringe 46 of an infusion configuration of the inventive blood purification system. During use, the infusion configuration may include a 10 to 20 mL air gap 18 within the infusing syringe 46 to allow for full volume evacuation all the way to a receiver syringe 48. The flange of the infusing syringe 46 is butted against the block of the syringe pump 46 to maintain the syringe at a 90° angle to allow for pooling of blood to minimize or eliminate bubbles in the blood.

FIG. 19 shows a receiving syringe 48 of a receiving configuration of the inventive blood purification system. A small air gap 18 is provided at the receiver syringe 48. The receiver syringe 48 dangles low to allow blood to flow from the infusing syringe 46 through the disposable blood purification assembly to the receiver syringe 48. Once all the blood has passed through the cuvette 12 subassembly, the pump 44 is stopped and the infusing syringe 46 plunger can then be manually pushed to purge the system all the way to the receiver syringe 48.

In accordance with an exemplary embodiment, the infusing blood source may include an infusing syringe 46 including a barrel for holding a volume of the blood. An orifice of the syringe 46 provides exit and entry into the barrel and a plunger is disposed within the barrel. The plunger may include a plunger flange for receiving pressure to move the plunger within the barrel for displacing the blood and forcing the blood out of the orifice. A connector may be disposed at the orifice. The infusing blood source can further include a pump 44 for controllably applying pressure to the plunger flange to move the plunger within the barrel to force the blood out of the orifice.

FIG. 20 illustrates a portable configuration of the inventive blood purification system. A light box housing with handles contains the inventive blood purification system. A roller pump may be installed within the housing that is easily accessible for tubing 22 lines. A cooling fan can be provided to remove excess heat generated by the radiation source 20 bulbs if necessary. An embodiment of the inventive blood purification system includes three irradiation chambers 14 with a radiation source 20 bulb in each of the irradiation chambers 14. In accordance with an alternative configuration, two radiation source 20 bulbs can be provided for each irradiation chamber 14 with a bulb on top and below where the disposable cuvette 12 is positioned between the bulbs. In accordance with the alternative configuration, a total of six bulbs are provided where the wattage of the blood purification system may be 300 W. Specular aluminum (for example, with a colzak or alzak specular aluminum finish) can be coated on the internal areas of the radiation chambers 14. Disposable 3 mm×4 mm cuvettes 12 may be provided for extracorporeal circulation of blood and exposure to UVC irradiation.

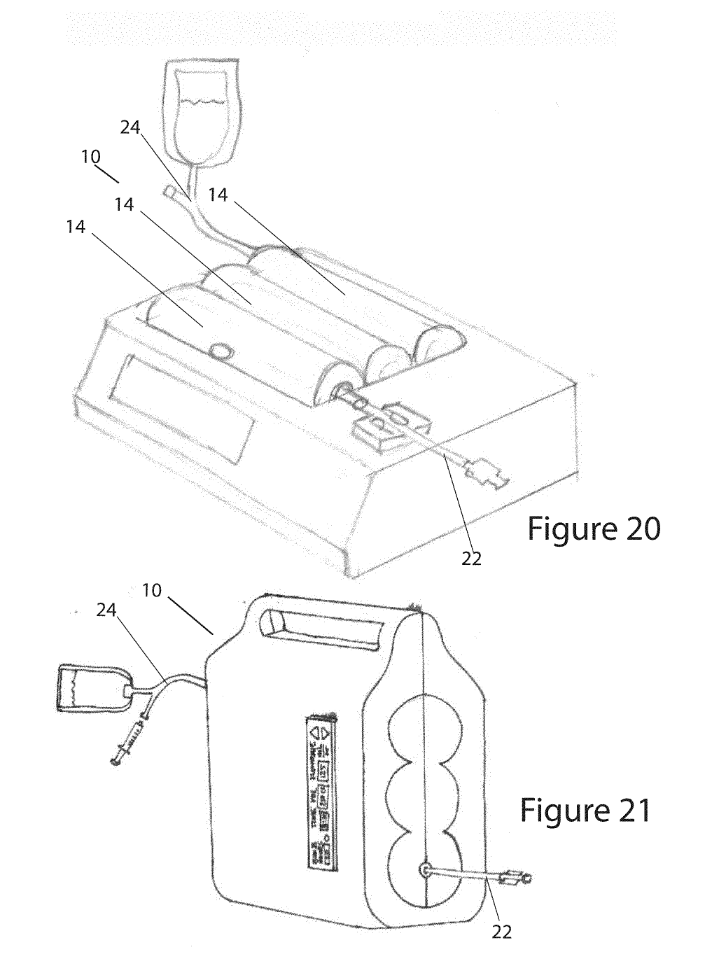

FIG. 21 illustrates the portable configuration of the inventive blood purification system housed in a portable housing. In accordance with the inventive method for reducing pathogens in blood, blood is supplied at a selectable flowrate from an infusion blood source through a blood transportation line 24 to a UV-passing cuvette 12. An irradiation chamber 14 is provided having a UV radiation source. The UV-passing cuvette 12 is fixed at a predetermined gap 18 from the UV radiation source. The blood within the UV-passing cuvette 12 is exposed directly to UV radiation emitted from the radiation source. The blood within the UV-passing cuvette 12 is also exposed indirectly to UV radiation emitted from the radiation source. The irradiation chamber 14 has a radiation recycling coating formed on surfaces of the irradiation chamber 14 for reflecting and recycling the UV radiation to expose the blood indirectly to the UV radiation emitted from the radiation source. The predetermined gap 18 and the flow rate are selected so that the direct and indirect exposure of the blood to the UV radiation kills pathogens in the blood while limiting the loss of healthy blood cells and blood constituents due to the UV radiation and heat generated by the UV radiation source.

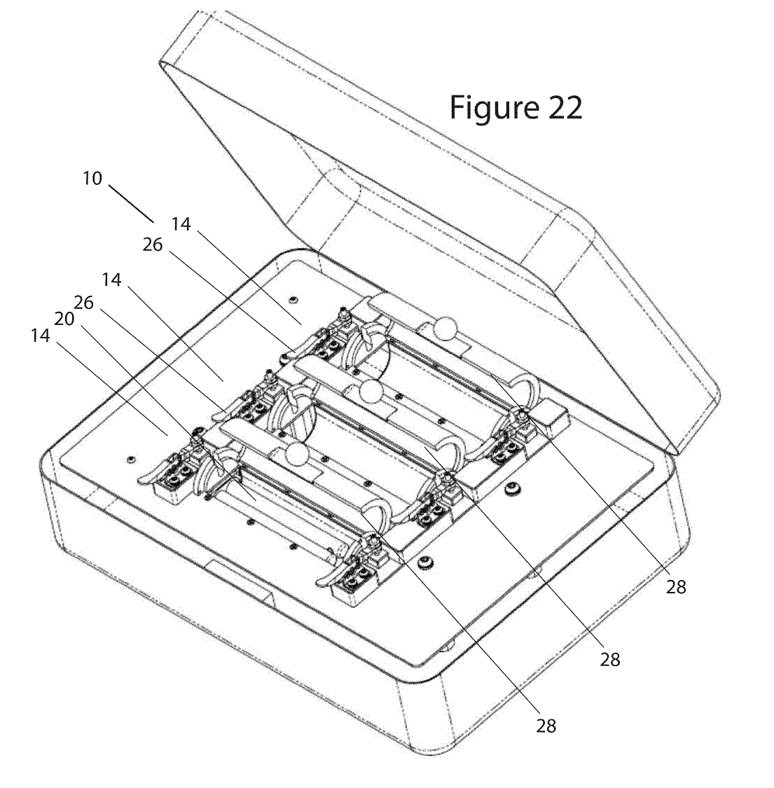

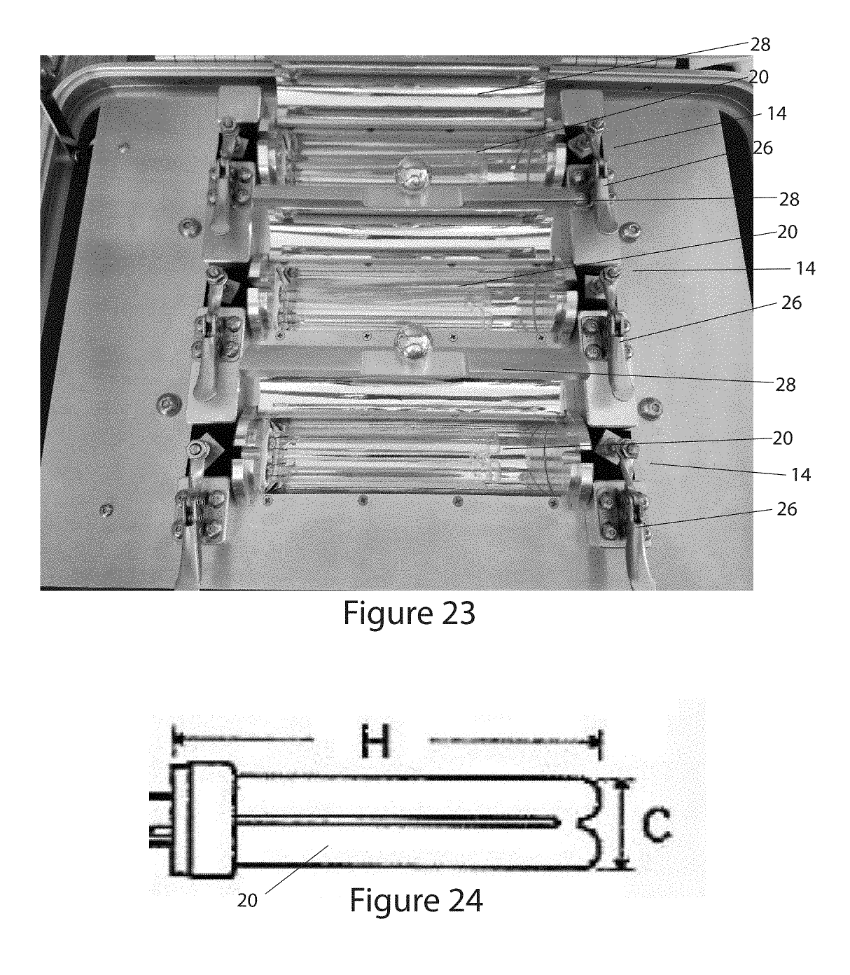

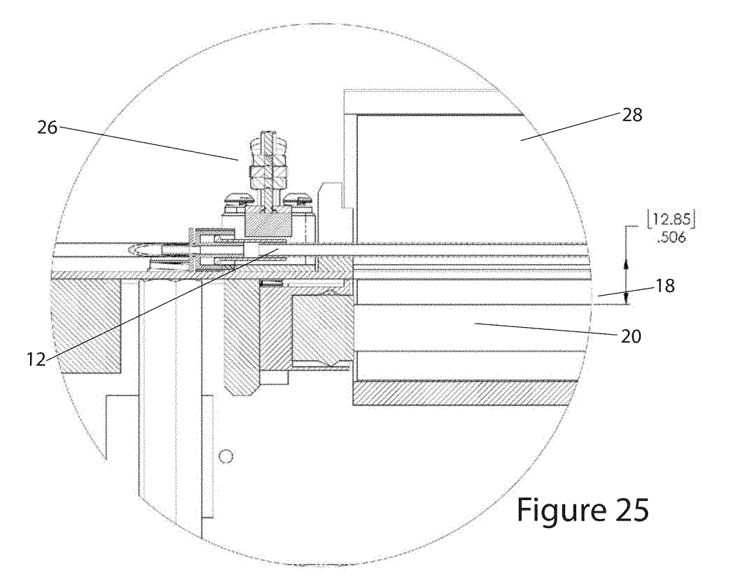

FIG. 22 is a perspective view of a higher intensity embodiment of the inventive blood purification system. FIG. 23 shows light chambers 14 of the embodiment of the inventive blood purification system shown in FIG. 22. FIG. 24 shows a UV radiation generating four pin twin bulb used in the embodiment of the inventive blood purification system shown in FIG. 22. FIG. 25 illustrates a gap 18 between a UV-passing cuvette 12 and the bulb used in the embodiment of the inventive blood purification system shown in FIG. 22. The predetermined gap 18 may be, for example, in the range of 12.85 mm or 0.5 inches.

In accordance with another aspect of the present invention, an apparatus is provided for reducing pathogens in blood. An infusing blood source provides a supply of blood at a selectable flowrate. A blood transportation line 24 transports the blood from the infusion blood source. A UV-passing cuvette 12 receives the blood from the blood transportation line 24. The UV-passing cuvette 12 is held in a first irradiation chamber 14 having a first UV radiation source. The first irradiation chamber 14 has a clamp 26 for fixing the UV-passing cuvette 12 at a predetermined gap 18 from the first UV radiation source 20 for exposing the blood within the UV-passing cuvette 12 directly to UV radiation emitted from the first radiation source. A radiation recycling coating is formed on surfaces of the first irradiation chamber 14 for reflecting and recycling the UV radiation to expose the blood indirectly to the UV radiation emitted from the radiation source. At least a second irradiation chamber 14 is provided having a second UV radiation source, second clamp for fixing a second UV-passing cuvette 12 at the predetermined gap 18 from the second UV radiation source 20 and the radiation recycling coating. The predetermined gap 18 and the flow rate are selected so that the direct and indirect exposure of the blood to the UV radiation kills pathogens in the blood while limiting the loss of healthy blood cells and blood constituents due to the UV radiation and heat generated by the UV radiation source.

Without in any way limiting the scope, interpretation, or application of the claims appearing below, a technical effect of one or more of the example embodiments disclosed herein is the recycling of the UV light provided by the bulb, along with the minimizing of the gap 18 between the bulb and the blood, which increases the efficiency of the system. This increased efficiency provides a beneficial cycle that enables faster throughput, higher UV radiation dosing to kill pathogens, and lower thermal exposure to the blood.

If desired, the different functions discussed herein may be performed in a different order and/or concurrently with each other. Furthermore, if desired, one or more of the above-described functions may be optional or may be combined.

Although various aspects of the invention are set out in the independent claims, other aspects of the invention comprise other combinations of features from the described embodiments and/or the dependent claims with the features of the independent claims, and not solely the combinations explicitly set out in the claims.

It is also noted herein that while the above describes example embodiments of the invention, these descriptions should not be viewed in a limiting sense. Rather, there are several variations and modifications which may be made without departing from the scope of the present invention as defined in the appended claims.

1) An apparatus for reducing pathogens in blood, comprising:
an infusing blood source for providing a supply of blood at a selectable flowrate;
a blood transportation line for transporting the blood from the infusion blood source;
a UV-passing cuvette for receiving the blood from the blood transportation line; and
an irradiation chamber having a UV radiation source, the irradiation chamber having a clamp for fixing the UV-passing cuvette at a predetermined gap from the UV radiation source for exposing the blood within the UV-passing cuvette directly to UV radiation emitted from the radiation source, and a radiation recycling coating formed on surfaces of the irradiation chamber for reflecting and recycling the UV radiation to expose the blood indirectly to the UV radiation emitted from the radiation source, wherein the predetermined gap and the flow rate are selected so that the direct and indirect exposure of the blood to the UV radiation kills pathogens in the blood while limiting the loss of healthy blood cells and blood constituents due to the UV radiation and heat generated by the UV radiation source.

2) The apparatus according to claim 1, wherein the UV-passing cuvette is a disposable quartz tube, and wherein the blood transportation line comprises a disposable flexible tubing for connecting with the UV-passing cuvette.

3) The apparatus according to claim 2, wherein the disposable flexible tubing has a length of substantially one meter to facilitate moving the blood back and forth through the irradiation chamber.

4) The apparatus according to claim 2, wherein the blood transportation line includes a blood bag spike for communicating with a blood bag; and wherein the infusing blood source includes a blood bag for containing the blood and a roller pump disposed on the blood transportation line for pumping the blood from the blood bag through the blood transportation line and to the UV-passing cuvette.

5) The apparatus according to claim 4, wherein the purified blood is reinfused into the patient.

6) The apparatus according to claim 2, wherein the infusing blood source includes a blood bag for containing the blood and a roller pump disposed on the blood transportation line for pumping the blood from the blood bag through the blood transportation line and to the UV-passing cuvette.

7) The apparatus according to claim 1, wherein the irradiation chamber further includes a cover having the radiation recycling coating for further recycling the radiation emitted from the radiation source to expose the blood indirectly to the UV radiation emitted from the radiation source.

8) The apparatus according to claim 1, further comprising at least one additional irradiation chamber.

9) The apparatus according to claim 1, wherein the infusing blood source includes an infusing syringe including a barrel for holding a volume of the blood, an orifice for providing exit and entry into the barrel and a plunger disposed within the barrel, the plunger including a plunger flange for receiving pressure to move the plunger within the barrel for displacing the blood and forcing the blood out of the orifice, and a connector disposed at the orifice, the infusing blood source further including a pump for controllably applying pressure to the plunger flange to move the plunger within the barrel to force the blood out of the orifice.

10) A method for reducing pathogens in blood, comprising:
supplying blood at a selectable flowrate from an infusion blood source through a blood transportation line to a UV-passing cuvette;
providing an irradiation chamber having a UV radiation source;
fixing the UV-passing cuvette at a predetermined gap from the UV radiation source;
exposing the blood within the UV-passing cuvette directly to UV radiation emitted from the radiation source, and exposing the blood within the UV-passing cuvette indirectly to UV radiation emitted from the radiation source, wherein the irradiation chamber has a radiation recycling coating formed on surfaces of the irradiation chamber for reflecting and recycling the UV radiation to expose the blood indirectly to the UV radiation emitted from the radiation source, and wherein the predetermined gap and the flow rate are selected so that the direct and indirect exposure of the blood to the UV radiation kills pathogens in the blood while limiting the loss of healthy blood cells and blood constituents due to the UV radiation and heat generated by the UV radiation source.

11) The method according to claim 10, wherein the UV-passing cuvette is a disposable quartz tube, and wherein the blood transportation line comprises a disposable flexible tubing for connecting with the UV-passing cuvette.

12) The apparatus according to claim 11, wherein the disposable flexible tubing has a length of substantially one meter to facilitate moving the blood back and forth through the irradiation chamber.

13) The method according to claim 11, wherein the blood transportation line includes a blood bag spike for communicating with a blood bag; and wherein the infusing blood source includes a blood bag for containing the blood and a roller pump disposed on the blood transportation line for pumping the blood from the blood bag through the blood transportation line and to the UV-passing cuvette.

14) The method according to claim 13, further comprising reinfusing the purified blood into the patient.

15) The method according to claim 11, wherein the infusing blood source includes a blood bag for containing the blood and a roller pump disposed on the blood transportation line for pumping the blood from the blood bag through the blood transportation line and to the UV-passing cuvette.

16) The method according to claim 10, wherein the irradiation chamber further includes a cover having the radiation recycling coating for further recycling the radiation emitted from the radiation source to expose the blood indirectly to the UV radiation emitted from the radiation source.

17) The method according to claim 10, further comprising at least one additional irradiation chamber.

18) The method according to claim 10, wherein the infusing blood source includes an infusing syringe including a barrel for holding a volume of the blood, an orifice for providing exit and entry into the barrel and a plunger disposed within the barrel, the plunger including a plunger flange for receiving pressure to move the plunger within the barrel for displacing the blood and forcing the blood out of the orifice, and a connector disposed at the orifice, the infusing blood source further including a pump for controllably applying pressure to the plunger flange to move the plunger within the barrel to force the blood out of the orifice.

19) An apparatus for reducing pathogens in blood, comprising:
an infusing blood source for providing a supply of blood at a selectable flowrate;
a blood transportation line for transporting the blood from the infusion blood source, the blood transportation line comprising a disposable flexible tubing;
a UV-passing cuvette for receiving the blood from the blood transportation line; and
a first irradiation chamber having a first UV radiation source, the first irradiation chamber having a first clamp for fixing the UV-passing cuvette at a predetermined gap from the UV radiation source for exposing the blood within the UV-passing cuvette directly to UV radiation emitted from the radiation source, and a radiation recycling coating formed on surfaces of the irradiation chamber for reflecting the recycling the UV radiation to expose the blood indirectly to the UV radiation emitted from the radiation source,
at least a second irradiation chamber having a second UV radiation source, second clamp for fixing a second UV-passing cuvette at the predetermined gap from the second UV radiation source and the radiation recycling coating, wherein the predetermined gap and the flow rate are selected so that the direct and indirect exposure of the blood to the UV radiation kills pathogens in the blood while limiting the loss of healthy blood cells and blood constituents due to the UV radiation and heat generated by the UV radiation source.

20) The apparatus according to claim 19, wherein the infusing blood source includes an infusing syringe including a barrel for holding a volume of the blood, an orifice for providing exit and entry into the barrel and a plunger disposed within the barrel, the plunger including a plunger flange for receiving pressure to move the plunger within the barrel for displacing the blood and forcing the blood out of the orifice, and a connector disposed at the orifice, the infusing blood source further including a pump for controllably applying pressure to the plunger flange to move the plunger within the barrel to force the blood out of the orifice.
